(19) United States
(12) Patent Application Publication
(10) Pub. No.: US 2011/0142905 A1
(43) Pub. Date: Jun. 16, 2011

(54) COATED TABLETS WITH REMAINING DEGRADATION SURFACE OVER THE TIME

(75) Inventors: Daniel Bar-Shalom, Kokkedal (DK); Kaisa Naelapää, Kobenhavn (DK)

(73) Assignee: BIONEER A/S, Horsholm (DK)

(21) Appl. No.: 13/058,933

(22) PCT Filed: Aug. 14, 2009
(86) PCT No.: PCT/DK09/50201

§ 371 Date: Feb. 14, 2011

Related U.S. Application Data
(60) Provisional application No. 61/088,859, filed on Aug. 14, 2008.

(51) Int. Cl. A61K 9/22; B05D 3/00; A61K 31/485; A61K 31/403; A61K 35/74; A61P 1/00; A61P 25/04

(52) U.S. Cl. 424/423; 424/468; 427/2.21; 424/422; 514/282; 514/411; 424/93.44

(57) ABSTRACT

The present invention relates to a pharmaceutical composition for controlled delivery of at least one active ingredient into an aqueous phase, said pharmaceutical composition comprising: a tablet, preferably obtainable by compression, said tablet comprising said at least one active ingredient and optionally excipients; and a coating, applied on said tablet, said coating covering at least part of said tablet to impede the release of said at least one active ingredient from at least part of the surface of said tablet, said coating being applied in a manner allowing the release of said at least one active ingredient from said tablet after contacting said pharmaceutical composition with said aqueous phase, establishing one or more degradation surfaces of said tablet; wherein the first derivative of the area of each degradation surface with respect to time is larger than or equal to zero.

### TECHNICAL FIELD OF THE INVENTION

The present invention relates to a pharmaceutical composition for controlled delivery. In particular the present invention relates to a coated multilayer tablet, which, after contact with an aqueous phase, release active pharmaceutical ingredient from the end surfaces in a controlled manner.

### BACKGROUND OF THE INVENTION

The International Patent Application WO 89/09066 discloses a pharmaceutical composition obtainable by extrusion, comprising a matrix of substantially water soluble crystalline polymer, such as polyglycol, at least one active substance, and optionally a surface active agent and/or a filler. This pharmaceutical composition is expensive to produce, as it requires a thermoplastic process. Further, the matrix and surrounding materials have to be thermoplastic materials, and they have to be thermoplastic compatible.

The background art comprises the manufacture of a hot-melt extrudable pharmaceutical composition dependent on the excipient polyglycol. The composition is made by extruding ingredients into a pre-formed silicone tube.

The U.S. Pat. No. 5,256,440 discloses a coated pharmaceutical composition with one or more grooves in the shape of circles on the top of the composition. According to the disclosure coating of the composition will provide a coat at the bottom of the grooves, which will be very thin or not present. Upon contact with an aqueous environment the coating inside the groove detaches, whilst the coating outside of the groove stays attached to the composition. The coat is impermeable to and insoluble in water. The use of e.g. swellable materials in the dosage form core is mentioned. The API is released with a changing erosion area over time.

The U.S. Pat. No. 5,342,627 discloses pharmaceutical compositions of atypical geometrical shapes and at least one release surface. The applied coat is water swellable, fluid impermeable and very slowly soluble. The total area of the erosion front is constant as a function of time. The compositions are shaped with concave surfaces and a thicker middle part, such that the diminishing size of the composition upon dissolution is compensated by the increased thickness of the composition towards the middle. Production and coating of these atypical compositions is non-trivial and requires special equipment.

The U.S. Pat. No. 4,839,177 discloses pharmaceutical compositions wherein the API is mixed with swellable and gellable excipients. Multilayered tablets are disclosed. The core is encircled by a coat insoluble in aqueous liquid leaving only one surface of the composition free of coat, thereby providing a tablet with one open end. Upon contact with an aqueous environment the uncoated part of the tablet swells, pushing off the coat and the API is released. The degree of swelling constitutes the primary control factor for the control of release of the API. The necessary coating equipment is expensive, and the coating procedure is time consuming.

The International Patent Application WO 2008/006216 discloses a round multilayered pharmaceutical composition comprising anti-inflammatory and cytoprotective agents. The composition is coated with a water impermeable coat that envelops the tablet core except for at least one exposed release face of at least one end of the core. The release face is substantially perpendicular to the longitudinal axis of the core. The dosage form can be designed to release the active pharmaceutical ingredients (API's) at variable time intervals at a steady rate. The different layers may comprise well known pharmaceutical excipients. The application of the coat covering only part of the pharmaceutical compositions demands specialized equipment e.g. a core-coater fitted with a specially designed tool for placing tablet cores precisely in the coating material. Equipment like this is expensive and the procedure of coating is time consuming.

The International Patent Application WO 03/007918 discloses a controlled release pharmaceutical composition comprising a core and a substantially insoluble coat covering 25-99% of the surface area of the tablet core. The coat is applied by means of electrostatic deposition of a powder to form a thin film. The exact location of the coat can be controlled. According to the disclosure, there are no needs for a special geometric shape. Further, WO 03/007919 discloses multilayered controlled release pharmaceutical compositions, wherein one face of the tablet core (in the longitudinal direction) is exposed to the aqueous environment, thereby achieving zero order release of one API. According to the disclosure, there are no needs for a special geometric shape. WO 03/007918 and WO 03/007919 disclose a coating process that is expensive and demand specialized equipment.

The International Patent Application WO 92/05776 discloses controlled released pharmaceutical compositions. The core has two parallel planar surfaces (i.e. the top and bottom) and a coat surrounding the core except for the planar surfaces (i.e. on all lateral surfaces). The coat is impermeable, i.e. substantially protecting the core from dissolution and covers the lateral tablet edge. The dosage form provides constant release of API by maintaining a constant surface area. The core is free of materials that cause swelling or disintegration which also imply that the API is free from having disintegrating properties. Various shapes or sizes of the core are possible as long as two opposing planar surfaces are available. This patent application discloses hand coating, by a brush, which is inherently an expensive and time consuming way of coating, which is hardly applicable on an industrial scale.

Hence, an improved pharmaceutical composition for the controlled delivery of an API with a coating that is easily produced on an industrial scale would be advantageous, and in particular a more efficient and/or reliable production method of such a pharmaceutical composition would be advantageous.

### SUMMARY OF THE INVENTION

Thus, an object of the present invention relates to providing a pharmaceutical dosage form.

In particular, it is an object of the present invention to provide a method for producing a pharmaceutical dosage form that solves the above mentioned problems of the prior art with expensive and cumbersome manufacturing.

Thus, one aspect of the invention relates to a pharmaceutical composition for controlled delivery of at least one active ingredient into an aqueous phase, said pharmaceutical composition comprising:
I. a tablet obtainable by compression, said tablet comprising said at least one active ingredient and optionally excipients;
II. a coating, applied on said tablet, said coating covering at least part of said tablet to impede the release of said at least one active ingredient from at least part of the surface of said tablet, said coating being applied in a manner allowing the release of said at least one active ingredient from said tablet after contacting said pharmaceutical composition with said aqueous phase, establishing one or more degradation surfaces of said tablet;
   wherein the derivative of the area, a, of each degradation surface, with respect to time, t, obeys:

$$\frac{\partial a}{\partial t} \geq 0 ,$$

after each degradation surface has been established, and/or during delivery of said at least one active ingredient.

Another aspect of the present invention relates to a pharmaceutical composition, for controlled delivery of at least one active ingredient into an aqueous phase, said pharmaceutical composition comprising:
I. a tablet, preferably obtainable by compression, said tablet comprising said at least one active ingredient and optionally excipients;
II. a coating, applied on said tablet, said coating covering at least part of said tablet to impede the release of said at least one active ingredient from at least part of the surface of said tablet, said coating being applied in a manner allowing the release of said at least one active ingredient from said tablet after contacting said pharmaceutical composition with said aqueous phase, establishing one or more degradation surfaces of said tablet;
   wherein the derivative of the area, a, of each degradation surface, with respect to time, t, obeys:

$$\frac{\partial a}{\partial t} \geq 0 ,$$

after each degradation surface has been established, and/or during delivery of said at least one active ingredient;
wherein said tablet has at least one sharp edge and a coating in proximity of said sharp edge, said coating being diminished in thickness or not present over said sharp edge.

Yet another aspect of the present invention is to provide a method for making a pharmaceutical composition for controlled delivery of at least one active ingredient into an aqueous phase, said method comprising:
I. producing a tablet by compression, comprising said at least one active ingredient and optionally excipients;
II. subsequently coating at least part of said tablet with at least one coating to impede the release of said at least one active ingredient from at least part of the surface of said tablet, said coating being applied in a manner allowing the release of said at least one active ingredient from said tablet after contacting said pharmaceutical composition with said aqueous phase, establishing one or more degradation surfaces of said tablet;
   wherein the derivative of the area, a, of each degradation surface, with respect to time, t, obeys:

$$\frac{\partial a}{\partial t} \geq 0 ,$$

after each degradation surface has been established, and/or during delivery of said at least one active ingredient.

Still another aspect of the present invention is to provide a method for making a pharmaceutical composition for controlled delivery of at least one active ingredient into an aqueous phase, said method comprising:
I. Producing a tablet, preferably by compression, said tablet comprising said at least one active ingredient and optionally excipients;
II. subsequently coating at least part of said tablet with at least one coating to impede the release of said at least one active ingredient from at least part of the surface of said tablet, said coating being applied in a manner allowing the release of said at least one active ingredient from said tablet after contacting said pharmaceutical composition with said aqueous phase, establishing one or more degradation surfaces of said tablet;
   wherein the derivative of the area, a, of each degradation surface, with respect to time, t, obeys:

$$\frac{\partial a}{\partial t} \geq 0 ,$$

after each degradation surface has been established, and/or during delivery of said at least one active ingredient;
and wherein said tablet has at least one sharp edge and a coating in proximity of said sharp edge, said coating being diminished in thickness or not present over said sharp edge.

### BRIEF DESCRIPTION OF THE FIGURES

FIG. 1 shows a coated monolayer cylindrical rod-shaped composition (1) with sharp edges and open ends. The coat (2) covers the entire tablet except for the open ends. The surface with the active substance for constant release is indicated in (3).

FIG. 2 shows a cross-section of an entirely coated cylindrical rod-shaped monolayer composition (4), with sharp edges (5), for the constant release of an active substance (3). The coat (2) covers the entire tablet. (6) shows an example of the diminished thickness of the coat covering one of the sharp edges (5).

FIG. 3 shows a cross-section of an entirely coated cylindrical rod-shaped monolayer composition (9) with beveled edges (7) for the constant release of an active substance (3). The coat (2) covers the entire tablet. (8) shows the diminished thickness of the coat covering the beveled edge (7). The beveled edge may thus also provide a thinner coating over the edge.

FIG. 4 shows a cross-section of a coated monolayer cylindrical rod-shaped composition for the constant release of an active substance (3). The composition comprises an active substance which is substantially homogeneously distributed in the tablet and is covered with a coating (2) which is open at both ends 10. The coating is substantially insoluble in and impermeable to fluids such as body fluids during the intended release period. The tablet may thus slowly erode from the open ends by the action of an aqueous medium in which the composition is employed, so that the surface area of the erosion front of the core) exposed to the aqueous phase remains substantially constant with time, whereby the active substance is released at a constant and controlled rate.

FIG. 5 shows a cross-section of the coated monolayer cylindrical rod-shaped composition shown in FIG. 4 after part of the core has been eroded. The tablet is covered with a coat (2) which is open at both ends (10). The surface area of the eroded erosion front of the core is indicated by (11). The tablet is thus slowly eroded from the open ends by the action of the aqueous medium in which the composition is employed, so that the surface area of the erosion front of the tablet (11) exposed to the aqueous phase remains substantially constant with time, whereby the active substance (3) is released at a constant and controlled rate.

FIG. 6 shows a cross-section of a three layered coated cylindrical rod-shaped composition for the constant release of an active substance (3). The tablet is covered with a coating (2) which is open at both ends (10). The composition comprises an active substance which is substantially homogeneously distributed in the middle layer of the core (3). Layers (12) constitute expansion layers. The coating is substantially insoluble in and impermeable to fluids such as body fluids during the intended release period. The tablet is thus slowly eroded from the open ends by the action of the aqueous medium in which the composition is situated, so that the surface area of the erosion front of the tablet exposed to the aqueous phase remains substantially constant with time, whereby the active substance is released at a constant and controlled rate.

FIG. 7 shows a cross-section of a seven layered coated cylindrical rod-shaped composition for the constant release of an active substance (layers 3). The tablet is covered with a coating (2) which is open at both ends (10). The composition comprises an active substance which is substantially homogeneously distributed in the layers (3) of the core. The layers (12) constitute expansion layers. The layers (13) are lag layers. The coating is substantially insoluble in and impermeable to fluids such as body fluids until all active ingredients have been released. This will provide for a pharmaceutical formulation with provides bursts of release of active ingredient.

FIG. 8 shows a cross-section of an entirely coated cylindrical rod-shaped monolayer composition (14), with sharp edges (5), for the constant release of an active substance (3). The coat (2) covers the entire tablet. (6) indicates an example of the diminished thickness of the coat covering one of the sharp edges (5). A thin layer (15) of an excipient, which is easily dissolvable in an aqueous phase, allows the end surface layers to be removed upon contact with the aqueous phase.

FIG. 9 shows a cross-section of an entirely coated cylindrical rod-shaped monolayer composition (16) with protruding edges (17) for the constant release of an active substance (3). The coat (2) covers the entire tablet. (8) shows the diminished thickness of the coat covering the protruding edge (17). The protruding edge may thus also provide a thinner coating over the edge.

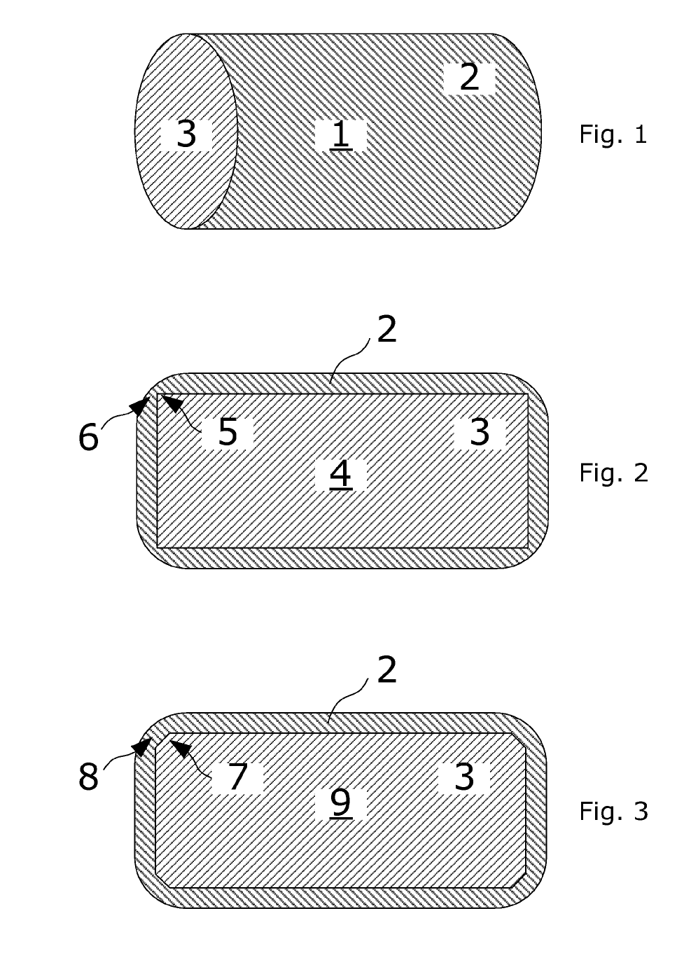

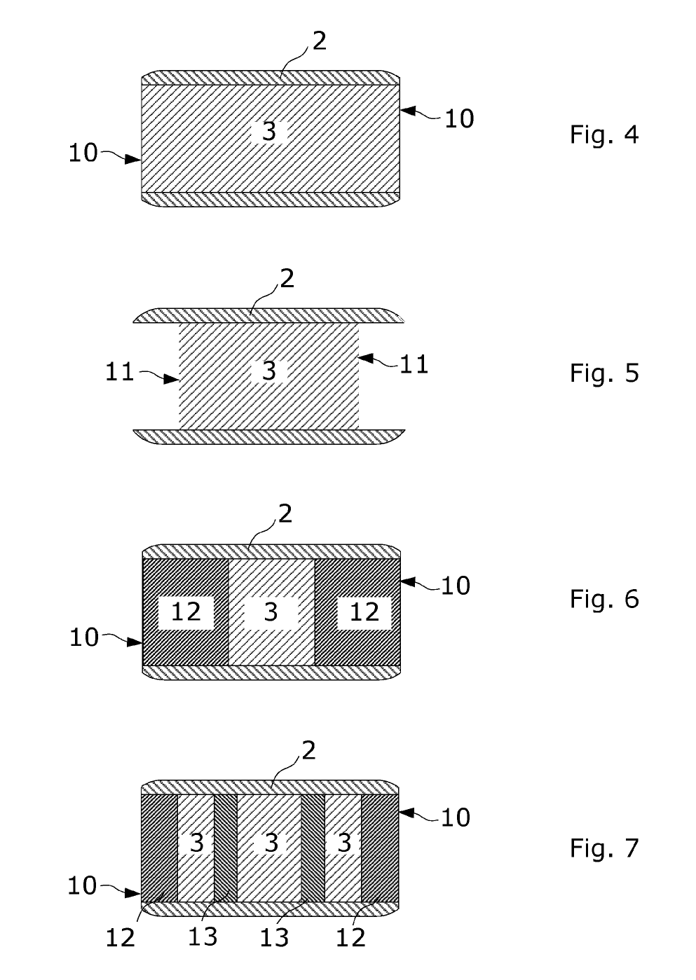

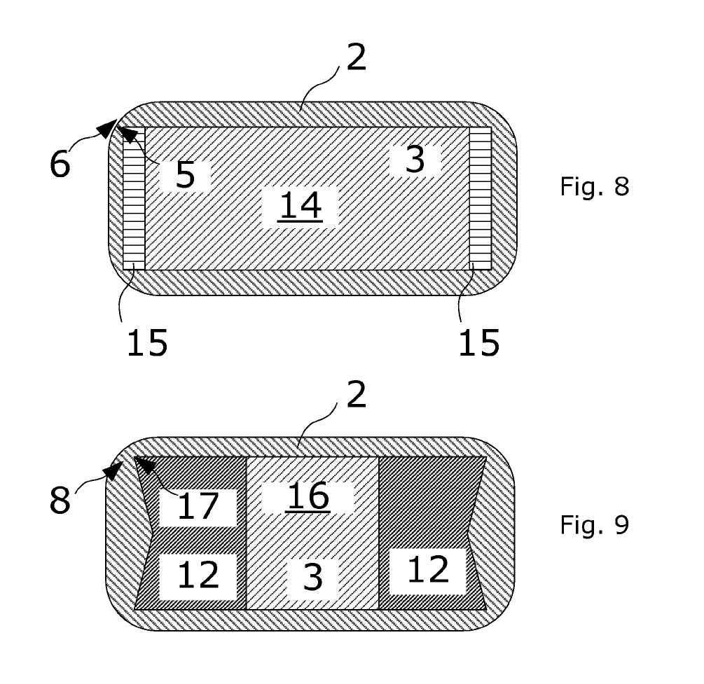

The present invention will now be described in more detail in the following.

### DETAILED DESCRIPTION OF THE INVENTION

According to an aspect, the present invention concerns a pharmaceutical composition for controlled delivery of at least one active ingredient into an aqueous phase, said pharmaceutical composition comprising:
I. a tablet obtainable by compression, said tablet comprising said at least one active ingredient and optionally excipients;
II. a coating, applied on said tablet, said coating covering at least part of said tablet to impede the release of said at least one active ingredient from at least part of the surface of said tablet, said coating being applied in a manner allowing the release of said at least one active ingredient from said tablet after contacting said pharmaceutical composition with said aqueous phase, establishing one or more degradation surfaces of said tablet;
   wherein the derivative of the area, a, of each degradation surface, with respect to time, t, obeys:

$$\frac{\partial a}{\partial t} \geq 0 ,$$

after each degradation surface has been established, and/or during delivery of said at least one active ingredient.

The active ingredient may be any pharmaceutically acceptable ingredient, i.e. any API.

The active ingredient may be released by diffusion and/or erosion and/or disintegration and/or dissolution and/or osmotic pressure equalization from said tablet.

Preferably the tablet is a compressed tablet. This provides for a tablet, which is easy and inexpensive to produce. The tablet may be compressed using a punch, such as a flat-faced, concave or convex punch. Compression may be performed without adding additional heat. The tablet may be produced without moulding, extrusion, heat melting or without the use of a thermoplastic process. Preferably the tablet is directly obtainable by compression.

Preferably the tablet is a solid, bulk object, preferably without any holes or apertures.

The release of active ingredient from the tablet is controlled by the composition of the tablet, its geometry and the coating. Hence, one of the factors determining the release is the cross section of the tablet in contact with the aqueous phase.

The degradation surfaces are established when contact between an aqueous phase and the tablet is established. This is the surface of the tablet at which the tablet is degraded and/or active ingredient is released.

According to an aspect, the area of each degradation surface is substantially constant after being established, and/or during delivery of said at least one active ingredient.

According to an aspect, the derivatives of the area, a, of each degradation surface, with respect to time, t, obeys:

$$\frac{\partial a}{\partial t} \geq 0 ,$$

after each degradation surface has been established, and/or during delivery of said at least one active ingredient. This means that the area of each degradation surface is substantially constant or growing after being established and/or during release of the active ingredient.

According to an aspect, the second derivative of the diameter, d, of each degradation surface, with respect to time, t, obeys:

$$\frac{{\partial }^{2} d}{\partial {t}^{2}} \leq 0 ,$$

after each degradation surface has been established, and/or during delivery of said at least one active ingredient. In case the degradation surface is growing, the growth rate is either substantially constant or decreasing with time. This may be obtained by a tablet with rounded or convex sides, having exposed ends.

The diameter of each degradation surface area is defined to be the largest distance that can be formed between two opposite parallel lines tangent to its boundary, no matter whether the shape of the surface area is plane, convex or concave. In case the degradation surface area has sharp corners, the diameter is defined to be the larger of the largest distance between two opposite parallel lines tangent to the boundary, if such parallel lines exists, or the largest distance between one corner and one opposite parallel line tangent to the boundary, or the largest distance between two opposite corners.

The expressions:

$$\frac{\partial a}{\partial t} \geq 0 and \frac{{\partial }^{2} d}{\partial {t}^{2}} \leq 0$$

does not necessarily mean that the derivatives are defined at all points, but rather that the area of each degradation surface is substantially constant or growing after being established and/or during release of the active ingredient, and in case the degradation surface is growing, the growth rate is either substantially constant or decreasing with time.

According to an aspect, the first derivative of the area of each degradation surface with respect to time is larger than or equal to zero, and the second derivative of the area of each degradation surface with respect to time is smaller than or equal to zero. In accordance with this, ∂a/∂t=0, expresses that the area of each degradation surface is substantially constant after being established, and/or during delivery of said at least one active ingredient. This may be obtained e.g. by a cylindrical tablet coated on the curved surface, said tablet having exposed, uncoated ends.

Most conventional tablets have ∂a/∂t<0. Such conventional tablets are eroded from all sides, and become gradually smaller, thus diminishing the area being eroded or through which the active ingredient is released.

The expression ∂a/∂t>0 shows that the tablet may be thicker in the middle, and the expression ∂²d/∂t²<0 shows that the tablet may be convex, e.g. have a rounded shape, making it easier to swallow, and allowing administering of a larger amount of active ingredient in the tablet. Similarly, the expression ∂a/∂t>0 shows that the tablet may be thicker in the middle, and have ∂²d/∂t²=0, such that the tablet may have plane sides. Increasing degradation surface with time may also compensate for otherwise decreasing diffusion rate of active ingredient, due to the effect of any remaining coating on the movement of a surrounding aqueous phase with respect to the degradation surface.

According to an aspect, the coating may be substantially water-impermeable and/or water impervious. According to an aspect, the coating is impervious to water a minimum 25% of the time it takes before 90% of the active ingredient is released. This may be advantageous e.g. when the active ingredient is to be absorbed in the small intestines.

The delivery rate of said at least one active ingredient into the aqueous phase is generally controlled by
a. the part of the surface of the tablet covered by the coating;
b. the character of the coating; and
c. the disintegration and/or diffusion and/or erosion and/or dissolution and/or osmotic pressure equalization speed of said tablet, allowing controlled delivery of said at least one active ingredient into the aqueous phase.

For compressed tablets the following notation may be used. A tablet face is the surface area formed by the punch tip. According to an aspect, a tablet face constitutes a degradation surface. A band is the area between the opposing tablet cup profiles or tablet faces. The die wall forms the tablet band. A land (the surface of a beveled edge) forms an angle with the tablet band and creates a junction between the band and cup profile. Edge chipping refers to the case wherein the tablets periphery chips during the coating process or during friability testing. A tablet may have a land to avoid edge chipping.

According to another aspect, the present invention concerns a pharmaceutical composition, for controlled delivery of at least one active ingredient into an aqueous phase, said pharmaceutical composition comprising:
III. a tablet, preferably obtainable by compression, said tablet comprising said at least one active ingredient and optionally excipients;
IV. a coating, applied on said tablet, said coating covering at least part of said tablet to impede the release of said at least one active ingredient from at least part of the surface of said tablet, said coating being applied in a manner allowing the release of said at least one active ingredient from said tablet after contacting said pharmaceutical composition with said aqueous phase, establishing one or more degradation surfaces of said tablet;
   wherein the derivative of the area, a, of each degradation surface, with respect to time, t, obeys:

$$\frac{\partial a}{\partial t} \geq 0 ,$$

after each degradation surface has been established, and/or during delivery of said at least one active ingredient;
wherein said tablet has at least one sharp edge and a coating in proximity of said sharp edge, said coating being diminished in thickness or not present over said sharp edge.

According to an aspect, the sharp edge may be or comprise a land.

According to an aspect, the present invention concerns a pharmaceutical composition, wherein said tablet is a multilayer tablet. The different layers have different compositions.

According to an aspect, the present invention concerns a pharmaceutical composition, wherein at least one layer, comprising at least part of said at least one active ingredient, is not in direct contact with said aqueous phase immediately after said one or more degradation surfaces have been established.

According to an aspect, the present invention concerns a pharmaceutical composition, wherein said tablet consist of a number of layers selected among 1, 2, 3, 4, 5, 6, 7, 8, 9, 10, 11, 12, 13, 14, 15, 16, 17, 18, 19, 20.

According to an aspect, the present invention concerns a pharmaceutical composition, wherein said tablet comprises at least 2 layers.

According to an aspect, the present invention concerns a pharmaceutical composition, wherein said tablet comprises at least 3 layers, two of the layers being positioned at opposite extremes of said tablet. The opposite extremes of a tablet may be each end of an elongated tablet.

According to an aspect, the present invention concerns a pharmaceutical composition, wherein said at least one coating partly covers the tablet such that no other part than the layers positioned at opposite extremes are in direct contact with the aqueous phase upon contacting said composition with an aqueous phase.

According to an aspect, the present invention concerns a pharmaceutical composition, wherein degradation of said tablet or release of said at least one ingredient occurs at one or two degradation surfaces.

According to an aspect, the present invention concerns a pharmaceutical composition, wherein the tablet has parallel sides, preferably between two degradation surfaces.

According to an aspect, the present invention concerns a pharmaceutical composition, wherein the shape of the tablet and/or the pharmaceutical composition is substantially cylindrical such as a right circular cylinder, a circular cylinder, an elliptic cylinder or a pseudo elliptical cylinder. The tablet and/or the pharmaceutical composition may be barrel or almond shaped. Some outwards curvature provide for a pharmaceutical formulation which is easier to swallow.

According to an aspect, the present invention concerns a pharmaceutical composition, wherein the shape of the tablet and/or the composition is a hexahedron, such as a parallelepiped, a rhombohedron, a trigonal trapezohedron, a cuboid or a cube.

According to an aspect, the present invention concerns a pharmaceutical composition, wherein each degradation surface is approximately constant in size.

According to an aspect, the pharmaceutical composition fulfils:

$$\frac{\partial a}{\partial t} = 0 and \frac{{\partial }^{2} d}{\partial {t}^{2}} = 0.$$

According to another aspect, the pharmaceutical composition fulfils:

$$\frac{\partial a}{\partial t} > 0 and \frac{{\partial }^{2} d}{\partial {t}^{2}} < 0.$$

According to an aspect, the present invention concerns a pharmaceutical composition, wherein the surface between the tablet and a surrounding aqueous phase, through which said at least one active ingredient is released, is approximately circular, elliptical, or quadrangular.

According to an aspect, the present invention concerns a pharmaceutical composition, wherein said tablet is obtainable by a process comprising compression, moulding, casting, extrusion, heat melting, melt casting, and/or a thermoplastic process. According to an aspect, the tablet may be obtained while heating.

According to an aspect, the present invention concerns a pharmaceutical composition, wherein said tablet is directly obtainable by compression. This may be the last step before coating.

According to an aspect, the present invention concerns a pharmaceutical composition, wherein said coating is applied by spray coating, pan coating, melt coating, fluid bed coating, cyclone coating, film coating, powder coating, or dip coating.

According to an aspect the invention concerns a method wherein the coating of tablets, preferably having sharp edges, is partly or completely removed during drying of the coated tablets due to the coated tablets hitting each other and/or at least one external container.

According to an aspect the invention concerns a method wherein said at least one coating is applied using a fluid bed. Preferably a fluid bed is used to abrade or remove sharp edges of the coated tablets, while other devices may be applied.

According to an aspect, the present invention concerns a pharmaceutical composition, wherein at least one surface area of said tablet is not covered by said at least one coating after said pharmaceutical composition has been brought into contact with the aqueous phase, thereby allowing contact between said tablet and the aqueous phase.

According to an aspect the invention concerns a method wherein said at least one coating is applied by alternately coating and drying the tablet, a number of times selected among 2, 3, 4, 5, 6, 7, 8, 9, 10 and more times. Preferably the coating-drying cycles are performed 2, 3, 4, or 5 times, more preferably 3 or 4 times.

According to an aspect, the present invention concerns a pharmaceutical composition, wherein the pharmaceutical composition is obtained by coating the whole surface of the tablet, followed by removing a part of the surface thereby allowing subsequent exposure of a part of the tablet to the aqueous phase. It may be economically attractive to cover the whole tablet, and afterwards remove unwanted parts of the coating.

According to an aspect, the present invention concerns a pharmaceutical composition, wherein the removal of a part of the surface occurs after contacting said pharmaceutical composition with an aqueous phase.

According to an aspect, the present invention concerns a pharmaceutical composition, wherein the removal of a part of the surface comprises application of mechanical means, such as cutting, planing, grinding, polishing, planishing, graining, or application of a laser; or application of chemical means such as an acid or a base. Other means such as heating, cooling; using an air stream or hitting, are also contemplated.

According to an aspect, the present invention concerns a pharmaceutical composition, wherein said tablet has at least one sharp edge.

By sharp edge is meant that a part of the tablet should be sufficiently uneven that upon coating, the coating will be diminished in thickness over or not present on this part of the tablet. This would hence cover an instance where a coating process provides a diminished thickness around at least one edge of said tablet. Often, sharp edges are avoided during tablet manufacture, in order to avoid chipping. However, sharp edges may be desirable, in order to obtain coating with varying thickness. The tablets may even be provided with a protruding part in order to ensure a coating of the tablet with uneven thickness.

According to an aspect, the present invention concerns a pharmaceutical composition, wherein the thickness of said at least one coating is diminished over said at least one sharp edge. The tablet is either not coated or the coating is thinnest, where said coating covers said sharp edge.

According to an aspect, the present invention concerns a pharmaceutical composition, wherein the thickness of said at least one coating is diminished in an area.

According to an aspect, the present invention concerns a pharmaceutical composition, wherein the area wherein the thickness of said at least one coating is diminished defines a substantially continuous loop area, such that the thickness of said coating within and outside of said loop is larger than the thickness of said coating of said loop area.

According to an aspect, the present invention concerns a pharmaceutical composition, wherein the coating within said loop area is approximately convex.

According to an aspect, the present invention concerns a pharmaceutical composition, wherein the coating within said loop area becomes separate from the rest of the pharmaceutical composition upon contact of the pharmaceutical composition with the aqueous phase. The tablet may be designed to have a coating, a part of which falls off upon contacting the pharmaceutical composition with an aqueous phase.

According to a particularly preferred aspect of the invention, the tablet has an edge and a coating, the coating being thinner or not present in an area over the edge, the area forming a substantially continuous loop, such that the part of the coating within said loop becomes separate from the tablet after contacting the pharmaceutical composition with an aqueous phase, thereby establishing a degradation surface.

According to an aspect, the present invention concerns a pharmaceutical composition, wherein the coating within said loop area expands or contracts upon contact with the aqueous phase.

According to an aspect, the present invention concerns a pharmaceutical composition, wherein the tablet comprises a disintegrant in close proximity with the coating within said loop area. The disintegrant may be used to expel part of the coating, thereby establishing a degradation surface.

According to an aspect, the present invention concerns a pharmaceutical composition, wherein the tablet comprises an excipient, which is easily dissolvable in the solvent of said coating, in close proximity with the coating, preferably within a loop area. As an example, the excipent may be sugar, and the coating may be water-based.

According to an aspect, the present invention concerns a pharmaceutical composition, wherein the contact area between said tablet and the aqueous phase is approximately constant during the delivery of said at least one active pharmaceutical.

According to an aspect, the present invention concerns a pharmaceutical composition, wherein the aqueous phase is Simulated Gastric Fluid or Simulated Intestinal Fluid according to USP 31.

According to an aspect, the present invention concerns a pharmaceutical composition, wherein the pharmaceutical composition further comprises an enteric coating covering at least part of said tablet. Preferably the enteric coating covers the whole tablet.

According to an aspect, the present invention concerns a pharmaceutical composition, wherein less than an amount selected among 1, 2, 3, 5, 10, and 15 weight % of said at least one active ingredient is delivered during testing for 30 minutes in a USP 31 Paddle Apparatus in Simulated Gastric Fluid. For testing in the USP 31 Paddle Apparatus 2, it is preferred to perform the testing at 100 rpm, in 900 ml liquid, which may alternatively be an aqueous buffer.

According to an aspect, the present invention concerns a pharmaceutical composition, wherein the rate of delivery of said at least one active ingredient is approximately zero order upon testing in a USP 31 Paddle Apparatus in Simulated Intestinal Fluid for a period of time selected among 1, 2, 3, 4, 5, 6, 8, 10 and 12 hours.

According to an aspect, the present invention concerns a pharmaceutical composition, wherein the maximum delivery rate of said at least one active ingredient is later than a time selected among 0.5, 1, 2, 3, 4, 5, 6, 7, 8, 9, 10, 11 and 12 hours upon testing in a USP 31 Paddle Apparatus in Simulated Intestinal Fluid.

According to an aspect, the present invention concerns a pharmaceutical composition, wherein the maximum delivery rate of said at least one active ingredient is before a time selected among 1, 2, 3, 4, 5, 6, 7, 8, 9, 10, 11, 12 and 18 hours upon testing in a USP 31 Paddle Apparatus in Simulated Intestinal Fluid.

According to an aspect, the present invention concerns a pharmaceutical composition, providing burst delivery of said at least one active pharmaceutical ingredient.

According to an aspect, the present invention concerns a pharmaceutical composition, wherein the testing in Simulated Intestinal Fluid is preceded by placing said pharmaceutical composition in an amount of time selected among 5, 10, 15, 30, 45 and 60 minutes in a USP 31 Paddle Apparatus (2) in Simulated Gastric Fluid.

According to an aspect, the present invention concerns a pharmaceutical composition, wherein the tablet comprises an erosion rate modifier.

According to an aspect, the present invention concerns a pharmaceutical composition, wherein the tablet comprises a disintegrant or lubricant.

According to an aspect, the present invention concerns a pharmaceutical composition, wherein the tablet comprises a number of different active pharmaceutical ingredients selected among 1, 2, 3, 4, 5, 6, 7, 8, 9, and 10.

Examples of such substances comprises, but are not limited to, hypnotics, sedatives, tranquilizers, anti-convulsants, musclerelaxants, analgesics, anti-inflammatory, anesthetics, anti-spasmodics, anti-ulcer-agents, anti-parasitics, anti-microbials, anti-fungal, cardiovascular agents, diuretics, cytostatics, anti-neoplastic agents, anti-viral agents, anti-glaucoma agents, anti-depressants, sympathomimetics, hypoglycaemics, diagnostic agents, anti-cough, physic energizers, anti-parkinson agents, local anesthetics, muscle contractants, anti-malarials, hormonal agents, contraceptives, anorexic, anti-arthritic, anti-diabetic, anti-hypertensive, anti-pyretic, anti-cholingergic, bronchodilator, central nervous system, inotropic, vasodilator, vasoconstrictor, decongestant, hematinic, electrolyte supplement, germicidal, parasympathetolytic, parasymphatethomimetic, antiemetic, psychostimulant, vitamin, beta-blockers, H-2 blocker, beta-2 agonist, counterirritants, coagulating modifying agents, stimulants, anti-hormones, API-antagonists, lipid-regulating agents, uricosurics, cardiac glycosides, ergot and derivatives thereof, expectorants, muscle relaxants, anti-histamines, purgatives, contrastmaterials, radiopharmaceuticals, imaging agents, anti-allergic agents, ion channel inhibitors and activators.

Examples of specific active substances comprise, but are not limited to, codeine, ethylmorphine, dextromethorphan, noscapine, pentoxiverine, acetylcysteine, bromhexine, epinephrine, isoprenaline, orciprenaline, ephedrine, fenoterol, riiterol, ipratropium, cholinetheophyllinate, proxiphylline, bechlomethasone, budesonide, deslanoside, digoxine, digitoxin, disopyramide, proscillaridin, chinidine, procainamide, mexiletin, flecamide, alprenolol, proproanolol, nadolol, pindolol, oxprenolol, labetalol, timolol, atenolol, pentaeritrityltetranitrate, isosorbiddinitrate, isosorbidmononitrate, niphedipin, phenylamine, verapamil, diltiazem, cyclandelar, nicotinylalcholhol, inositolnico-tinate, alprostatdil, etilephrine, prenalterol, dobutamine, dopamine, dihydroergotamine, guanetidine, betanidine, meth Idopa, reserpine, guanfacine, trimethaphan, hydralazine, dihydralazine, prazosine, diazoxid, captopril, nifedipine, enalapril, nitroprusside, bendroflumethiaziede, hydrochlorthiazide, metychlothiazide, polythiazide, chlorthalidon, cinetazon, clopamide, mefruside, metholazone, bumetanide, ethacrynacide, spironolactone, amiloride, chlofibrate, nicotinic acid, nicheritrol, brompheniramine, cinnarizine, dexchlorpheniramine, clemastine, antazoline, cyproheptadine, promethazine, cimetidine, ranitidine, sucralfat, papaverine, moxaverine, atropin, butylscopolamin, emepron, glucopyrron, hyoscyamine, mepensolar, methylscopolamine, oxiphencyclimine, probanteline, terodilin, sennaglycosides, sagradaextract, dantron, bisachodyl, sodiumpicosulfat, etulos, diphenolxylate, loperamide, salazosulfapyridine, pyrvin, mebendazol, dimeticon, ferrofumarate, ferrosuccinate, ferritetrasemisodium, cyanochobalamine, folic acid heparin, heparin co-factor, diculmarole, warfarin, streptokinase, urokinase, factor VIII, factor IX, vitamin K, thiotepa, busulfan, chlorambucil, cyclophosphamid, melfalan, carmustin, mercaptopurin, thioguanin, azathioprin, cytarabin, vinblastin, vinchristin, vindesin, procarbazine, dacarbazine, lomustin, estramustin, teniposide, etoposide, cisplatin, amsachrin, aminogluthetimid, phosphestrol, medroxiprogresterone, hydroxiprogresterone, megesterol, noretisteron, tamoxiphen, ciclosporin, sulfisomidine, bensylpenicillin, phenoxymethylpenicillin, dicloxacillin, cloxacillin, flucloxacillin, ampicillin, amoxicillin, pivampicillin, bacampicillin, piperacillin, ezlocillin, mecillinam, pivmecillinam, cephalotin, cephalexin, cephradin, cephydroxil, cephaclor, cefuroxim, cefotaxim, ceftazidim, cefoxitin, aztreonam, imipenem, cilastatin, tetracycline, lymecycline, demeclocycline, metacycline, oxitetracycline, doxycycline, chloramphenicol, spiramycin, fusidic acid, lincomycin, clindamycin, spectinomycin, rifampicin, amphotericin B, griseofulvin, nystatin, vancomycin, metronidazole, tinidazole, trimethoprim, norfloxacin, salazosulfapyridin, aminosalyl, isoniazid, etambutol, nitrofurantoin, nalidixic acid, metenamine, chloroquin, hydroxichloroquin, tinidazol, ketokonazol, acyclovir, interferon idoxuridin, retinol, tiamin, dexpantenol, pyridoxin, folic acid, ascorbic acid, tokoferol, phytominadion, phenfluramin, corticotropin, tetracosactid, tyrotropin, somatotropin, somatrem, vasopressin, lypressin, desmopressin, oxytocin, chloriongonadotropin, cortison, hydrocortison, fludrocortison, prednison, prednisolon, fluoximesteron, mesterolon, nandrolon, stanozolol, oximetolon, cyproteron, levotyroxin, liotyronin, propylthiouracil, carbimazol, tiamazol, dihydrotachysterol, alfacalcidol, calcitirol, insulin, tolbutamid, chlorpropamid, tolazamid, glipizid, glibenclamid, phenobarbital, methyprylon, pyrityldion, meprobamat, chlordiazepoxid, diazepam, nitrazepam, oxazepa, dikaliumchlorazepat, lorazepam, flunitrazepam, alprazolam, midazolam, hydroxizin, chlomethiazol, propionmazine, alimemazine, chlorpromazine, levomepromazine, acetophenazine, fluphenazine, perphenazine, prochlorperazine, trifluoperazine, dixyrazi-ne, thioridazine, periciazin, chloprothixene, zuclopentizol, flupentizol, thithixen, haloperidol, trimipramin, opipramol, chlomipramin, desipramin, lofepramin, amitriptylin, nortriptylin, protriptylin, maptrotilin, caffeine, cinnarizine, cyclizine, dimenhydinate, meclozine, proetazine, thiethylperazine, metoclopramide, scopolamine, phenobarbital, phenyloine, ethosuximide, primidone, carbamazepine, chlonazepam, orphenadrine, atropine, bensatropine, biperiden, metixene, procylidine, levodopa, bromocriptin, amantadine, ambenon, pyridostigmine, synstigmine, disulfuram, morphine, codeine, pentazocine, buprenorphine, pethidine, phenoperidine, phentanyl, methadone, piritramide, dextropropoxyphene, ketobemidone, acetylsalicylic acid, phenazone, phenylbutazone, azapropazone, piroxicam, ergotamine, dihydroergotamine, cyproheptadine, pizitifen, flumedroxon, allopurinol, probenecid, sodiummaurothiomalate auronofin, penicillamine, estradiol, estradiolvalerianate, estriol, ethinylestradiol, dihydrogesteron, lynestrenol, medroxiprogresterone, noretisterone, cyclophenile, clomiphene, levonorgestrel, mestranol, ornidazol, tinidazol, ekonazol, chlotrimazol, natamycine, miconazole, sulbentin, methylergotamine, dinoprost, dinoproston, gemeprost, bromocriptine, phenylpropanolamine, sodium-chromoglicate, azetazolamide, dichlophenamide, betacarotene, naloxone, calciumfolinate, in particular clonidine, theophlline, dipyradamol, hydrochlorthiazide, scopolamine, indomethacine, furosemide, potassium chloride, morphine, ibuprofen, salbutamol, terbutalin.

Particularly preferred active ingredients are selected among thalidomide and tetrabenazine.

The active pharmaceutical ingredients can be in various forms, such as uncharged molecules, molecular complexes, a pharmacologically acceptable salt such as a hydro-chloride, hydrobromide, sulfate, laurylate, palmitate, phosphate, nitrite, nitrate, borate, acetate, maleate, tartrate, oleate, and salicylate. For acid active pharmaceutical ingredients, salts of metals, amines amino acids or organic cations, quaternary ammonium, can be used. Derivatives of active pharmaceutical ingredients such as esters, ethers and amides which have solubility characteristics suitable for use herein can be used alone or mixed with other active pharmaceutical ingredients. After release of the derivative from the composition it may be converted by enzymes, hydrolyzed by body pH or other metabolic processes to the parent active pharmaceutical ingredients or to another biologically active form.

The pharmaceutical composition is in addition suitable for the delivery of polypeptides, for example hormones such as growth hormones, enzymes such as lipases, proteases, carbohydrates, amylases, lactoferrin, lactoperoxidases, lysozymes, nanoparticles, etc., and antibodies. The composition may also be employed for the delivery of microorganisms, either living, attenuated or dead, for example bacteria, e.g. gastrointestinal bacteria such as streptococci, e.g. S. faecium, Bacillus spp. such as B. subtilis and B. licheniformis, lactobacteria, Asper-gillus spp., bifidogenic factors, or viruses such as indigenous vira, enterovira, bacteriophages, e.g. as vaccines, and fungi such as baker's yeast, Saccharomyces cerevisiae and fungi imperfecti. The composition may also be used for the delivery of active agents in specialized carriers such as liposomes, cyclodextrines, nanoparticles, micelles and fats.

A further use for which the composition of the invention is suited is the delivery of active substances to animals. Examples of such active substances for veterinary use are antiparasitics, corticosteroids, antibiotics, antiinflammatory agents, growth promoters and permittants, antifungals and antihelmintics. According to an aspect, the present invention concerns a pharmaceutical composition, wherein said tablet comprises an excipient selected among starch, lactose, magnesium stearate, microcrystalline cellulose, stearic acid, calcium phosphate, glycerol monostearate, sucrose, polyvinylpyrrolidone, gelatin, methylcellulose, sodium carboxymethylcellulose, sorbitol, mannitol, polyethylene glycol, dicalcium phosphate, calcium sulfate, lactose or sucrose or other disaccharides, cellulose, cellulose derivatives, kaolin, dry starch, other raonosaccharides, dextrin or other polysaccharides, inositol or mixtures thereof; binders such as acacia, sodium alginate, starch, gelatin, saccharides (including dextrose and lactose), molasses, extract of Irish moss, panwar gum, ghatti gum, mucilage of isapol husk, carboxymethylcellulose, methylcellulose, veegum, larch arabolactan, polyethylene glycols, ethylcellulose, water, alcohols, waxes, polyvinylpyrrolidone or mixtures thereof; lubricants such as talc, magnesium stearate, calcium stearate, stearic acid, hydrogenated vegetable oils, sodium benzoate, sodium chloride, leucine, carbowax 4000, magnesium lauryl sulfate, colloidal silicon dioxide and mixtures thereof; swellable polymers, including but not limited to, crosslinked polymethacrylate and polyacrylate polymers derivatized with hydroxyalkyl and/or ionizable acidic or basic functional groups, and their respective salt forms; crosslinked polyvinylpyrrolidone; crosslinked polyvinyl alcohols; poly(ethylene oxide)s; polymethacrylamides and polyacrylamides; derivatized or modified cellulosic polymers such as crosslinked sodium carboxymethylcellulose, crosslinked hydroxypropylcellulose, starch graft copolymers, crosslinked hydroxypropylmethylcellulose, crosslinked dextrans and agarose, and microcrystalline cellulose; carboxymethylamide; and polyelectrolytes.

Examples of disintegrants comprise, but are not limited to, starches, clays, cellulose derivatives including croscarmellose, gums, algins, various combinations of hydrogencarbonates with weak acids (e.g. sodium hydrogencarbonate/tartaric acid or citric acid) crospovidone, sodium starch glycolate, agar, cation exchange resins, citrus pulp, veegum HV, natural sponge, bentonite or mixtures there of; volatile solvents such as alcohols, including aqueous alcohols, petroleum benzine, acetone, ether or mixtures thereof; plasticizers such as sorbitol and glycerine; and others such as cocoa butter, polyethylene glycols, and mixtures thereof, hydrogenated vegetable oils, glycerinated gelatin or mixtures thereof.

Furthermore, the compositions may comprise one or more agents selected from the group consisting of sweetening agents, flavouring agents and colouring agents, in order to provide an elegant and palatable preparation. Examples of colouring agents are water soluble FD and C dyes and mixtures thereof with corresponding lakes and direct compression sugars such as Di-Pac from Amstar. In addition, coloured dye migration inhibitors such as tragacanth, acacia or attapulgite talc may be added.

According to an aspect, the present invention concerns a pharmaceutical composition, wherein said coating comprises a material selected among eudragits, acrylates, cellulose derivatives, ethyl cellulose, polyethylene, polyethylene oxide, polymethane, polyurethanes, including Estane F 30, butadiene-styrene block copolymers, including Kraton®, polyesters, silicone, rubber, gums, latex, shell lac, glass, ceramics, waxes, carnauba wax, paraffin and mixtures of thereof. Preferred coating substances are eudragits, acrylates and cellulose deivatives.

The coating is preferably substantially insoluble in water and/or impervious to water.

Examples of coating materials which disintegrate or crumble after release of the active pharmaceutical ingredient are cellulose acetate, polyamide, polyethylene, polyethylene terephtalate, polypropylene, polyurethane, including Estane F 30, polyvinyl acetate, polyvinyl chloride, silicone rubber, latex, polyhydroxybutyrate, polyhydroxyvalerate, teflon, polylactic acid or polyglycolic acid and copolymers thereof, copolymers such as ethylene vinyl acetate (EVA), styrene-butadiene-styrene (SBS) and styrene-isoprene-styrene (SIS), and long chain fatty acids having 12 to 20 carbon atoms, such as lauric acid, myristic acid, palmitic acid, stearic acid and arachidic acid.

In order to influence the properties of the coating material (e.g. the erosion rate, the strength of the coating and/or the ability to disintegrate or crumble), the coating may include one or more excipients such as diluents such as dicalcium phosphate, calcium sulfate, lactose or sucrose or other disaccharides, cellulose, cellulose derivatives, kaolin, mannitol, dry starch, glucose or other monosaccharides, dextrin or other polysaccharides, sorbitol, inositol or mixtures thereof; binders such as acacia, sodium alginate, starch, gelatin, saccharides (including glucose, sucrose, dextrose and lactose), molasses, extract of irish moss, panwar gum, ghatti gum, mucilage of isapol husk, carboxymethylcellulose, methylcellulose, veegum, larch arabolactan, polyethylene glycols, ethylcellulose, water, alcohols, waxes, polyvinylpyrrolidone or mixtures thereof; lubricants such as talc, magnesium stearate, calcium stearate, stearic acid, hydrogenated vegetable oils, sodium benzoate, sodium chloride, leucine, carbowax 4000, magnesium lauryl sulfate, colloidal silicon dioxide and mixtures thereof; disintegrants such as starches, clays, cellulose derivatives including croscarmellose, gums, algins, various combinations of hydrogencarbonates with weak acids (e.g. sodium hydrogencarbonate/tartaric acid or citric acid) crospovidone, sodium starch glycolate, agar, cation exchange resins, citrus pulp, veegum HV, natural sponge, bentonite or others mentioned above or mixtures thereof; volatile solvents as e.g. alcohols, aqueous alcohols, petroleum benzine, acetone, ether or mixtures thereof; plasticizers as e.g. sorbitol, glycerine, polyethylene glycols and mixtures thereof; others such as cocoa butter, polyethylene glycols and mixtures thereof, hydrogenated vegetable oils, glycerinated gelatin or mixtures thereof. Furthermore, excipients such as dextrin, sucralfate, calcium hydroxylapatite, calcium phosphate and fatty acids salts such as magnesium stearate may be included.

According to an aspect the invention concerns a method wherein said at least one coating comprises a plasticizer.

According to an aspect the invention concerns a method wherein said at least one coating does not comprise a plasticizer.

According to an aspect the invention concerns a method wherein said at least one coating comprises an amount of plasticizer optimised according to the desired release properties of the obtained pharmaceutical formulation.

The coated pharmaceutical composition may furthermore be enteric coated in cases where the API is sensitive to the environment in the stomach, e.g. when the API is degraded by gastric acid, or where it is undesirable for therapeutic reasons to expose the stomach to API, e.g. when the API causes irritation of the gastric mucosa. Furthermore, it may be desirable to target the release to a given segment in the intestines. This can be done because the passage time through the small intestine is relatively constant (4-6 hours), while the passage time through the stomach is dependent on numerous factors and therefore is rather unpredictable.

Materials suitable for enteric coating include cellulose acetate phthalate, formalin-treated gelatin, polyvinyl acetate phthalate, hydroxypropyl methylcellulose phthalate, methacrylic acid, methacrylic acid ester copolymers and mixtures thereof.

Coatings to improve the appearance and or taste, e.g. film or sugar coating, may be applied to the composition.

According to an aspect, the present invention concerns a pharmaceutical composition, for human or animal use. According to certain aspects, it may provide for a release during up to several months, e.g. if the composition is implanted.

According to an aspect, the present invention concerns a pharmaceutical composition, for pain relief, such as chronic pain, treatment of central nervous system disorders, birth control, fertility treatment, or any other related diseases.

According to an aspect, the present invention concerns a pharmaceutical composition, wherein said at least one ingredient is selected among antibiotics, minerals, or vitamins.

According to an aspect, the present invention concerns a pharmaceutical composition, for intracavity administration, such as oral, rectal, or vaginal administration. Preferably the pharmaceutical composition is for oral use.

According to an aspect, the present invention concerns a pharmaceutical composition which may advantageously be administered orally, rectally, vaginally, buccally, nasally, dermally, intraperitoneally or administered to a body cavity (e.g. the urinary bladder, kidney pelvis, the gall bladder, the pancreas, the uterus, a central nervous system cavity, infectious/malignant/post-operative cavities); as an implantate; and/or as an injectable tablet.

According to an aspect, the present invention concerns a method for making a pharmaceutical composition for controlled delivery of at least one active ingredient into an aqueous phase, said method comprising:
V. producing a tablet by compression, comprising said at least one active ingredient and optionally excipients;
VI. subsequently coating at least part of said tablet with at least one coating to impede the release of said at least one active ingredient from at least part of the surface of said tablet, said coating being applied in a manner allowing the release of said at least one active ingredient from said tablet after contacting said pharmaceutical composition with said aqueous phase, establishing one or more degradation surfaces of said tablet;
   wherein the derivative of the area, a, of each degradation surface, with respect to time, t, obeys:

$$\frac{\partial a}{\partial t} \geq 0 ,$$

after each degradation surface has been established, and/or during delivery of said at least one active ingredient.

According to an aspect, the present invention concerns a method for making a pharmaceutical composition for controlled delivery of at least one active ingredient into an aqueous phase, said method comprising:
I. Producing a tablet, preferably by compression, said tablet comprising said at least one active ingredient and optionally excipients;
II. subsequently coating at least part of said tablet with at least one coating to impede the release of said at least one active ingredient from at least part of the surface of said tablet, said coating being applied in a manner allowing the release of said at least one active ingredient from said tablet after contacting said pharmaceutical composition with said aqueous phase, establishing one or more degradation surfaces of said tablet;
   wherein the derivative of the area, a, of each degradation surface, with respect to time, t, obeys:

$$\frac{\partial a}{\partial t} \geq 0 ,$$

after each degradation surface has been established, and/or during delivery of said at least one active ingredient;
and wherein said tablet has at least one sharp edge and a coating in proximity of said sharp edge, said coating being diminished in thickness or not present over said sharp edge.

According to an aspect, the invention concerns a method, wherein the second derivative of the diameter, d, of each degradation surface, with respect to time, t, obeys:

$$\frac{{\partial }^{2} d}{\partial {t}^{2}} \leq 0 ,$$

after each degradation surface has been established, and/or during delivery of said at least one active ingredient.

According to an aspect, the invention concerns a method, wherein said at least one coating is manufactured as a tube, and said tablet is placed inside said tube, preferably by pushing said tablet into said tube and/or by pulling said tube over said tablet.

According to an aspect, the present invention concerns a method for making a pharmaceutical composition, wherein said at least one coating is a fluid at the time of application. Usually, the fluid would be a liquid or a gas. However, the invention also encompasses cases wherein solid microparticles are dispersed in a liquid or gas, and hence have flow properties as a fluid at the time of coating. According to other aspects, the coating is a liquid and/or a gas at the time of coating.

According to an aspect, the invention concerns a method, wherein a part of the surface of said tablet comprises an excipient, which is not compatible with said coating material, thereby rendering the tablet partly coated upon coating of the tablet. As an example, Eudragit may be used to coat surfaces, comprising Dimethicone (polydimethylsiloxane).

According to an aspect, the present invention concerns a pharmaceutical composition obtainable according to a method according to the invention.

According to an aspect, the present invention concerns a pharmaceutical composition according to or obtainable according to any other aspect of the invention, wherein said tablet comprises an active pharmaceutical ingredient selected among morphine, hydrocodone, oxycodone, hydromorphone, and carvedilol, as well as pharmaceutically acceptable salts thereof.

It should be noted that embodiments and features described in the context of one of the aspects of the present invention also apply to the other aspects of the present invention.

All patent and non-patent references cited in the present application are hereby incorporated by reference in their entirety.

The invention will now be described in further details in the following non-limiting contemplated examples.

Unless otherwise mentioned all percentages are weight/weight.

### EXAMPLES

General method of manufacture of controlled release pharmaceutical dosage form according to the present invention

In its simplest form, the invention discloses a tablet directly obtainable by compression on a single-punch machine by using a flat-faced tablet die punch to produce a circular cylindrical tablet core with at least one sharp edge. Prior to compression the powder has been thoroughly mixed. For some excipients granulation may be necessary before compression. The tablet is coated by conventional methods to leave a coat that covers the entire tablet said coat being diminished in thickness or not present over said at least one sharp edge.

### Example 1

### Preparation of a Coated Controlled Release Pharmaceutical Composition Comprising the Active Substance Carvedilol

### (I) Ingredients (Weight %) for the Slow Release Tablet Core Comprising API:

| | |
|---|---|
| Crosslinked hydroxypropylmethylcellulose | 55.6 |
| Methylcellulose | 9 |
| Carvedilol | 30 |
| Citric acid | 5.4 |

The ingredients (I) are blended for 15 minutes in a suitable blender followed by direct compression at 2500 psi for 30 seconds on a Carver Press with a conventional circular flat faced tablet punch. The tablet is coated in a conventional coating pan with a 20% w/w solution of polyvinyl chloride in tetrahydrofuran (IHF). This may provide a pharmaceutical composition with approximately zero order release of the API.

### Example 2

In its simplest form a mulitilayered coated controlled release pharmaceutical dosage form according to this invention comprise a tablet with two layers. One layer comprises API and excipients and the other layer comprises excipients. The tablet is obtained by direct compression in a single-punch machine by using a flat-faced tablet die punch to produce a circular cylindrical tablet core with at least one sharp edge. Each layer is filled successively into the tablet machine i.e. one layer is filled, then slightly compressed followed by retraction of the punch. Then the next layer of the tablet is filled into the machine followed by a final compression step. Prior to compression the different ingredients constituting each layer have been thoroughly blended. For some excipients granulation may be necessary before compression. The resulting tablet is coated by conventional methods to leave a coat that covers the entire tablet said coat being diminished in thickness or not present over said at least one sharp edge.

### Preparation of a Three Layered Coated Controlled Release Pharmaceutical Composition Comprising the Active Substance Carvedilol

The first layer comprises excipients that cause expansion, the second layer (the middle layer) of carvedilol and excipients and the third layer of excipients that cause expansion. See FIG. 6 for an example of the distribution of the different layers.

(II) Total Ingredients for the Tablet Layers Comprising Excipients that Cause Expansion (Layers One and Three):

| | |
|---|---|
| Microcrystalline cellulose (particle size 20-100 μM) | 60 |

### (III) Ingredients (for the Controlled Release Tablet Layer Comprising API (Layer Two):

| | |
|---|---|
| Crosslinked hydroxypropylmethylcellulose | 7.9 |
| Methylcellulose | 1.3 |
| Carvedilol | 30 |
| Citric acid | 0.8 |

The ingredients from (II) and (III) are blended separately for 15 minutes in a suitable blender followed by direct compression. Each type of layer is filled successively into the tablet machine followed by a short and soft compression before another layer is filled into the tablet machine. When the final layer is filled into the tablet machine the whole tablet is pressed at 2500 psi for 30 seconds on a Carver Press with a conventional circular flat faced tablet punch. The tablet is coated in a conventional coating pan with a 20% w/w solution of polyvinylchloride in tetrahydrofuran (IHF). This may provide a pharmaceutical composition with approximately zero order release of the API.

### Example 3

### Preparation of a Coated Controlled Release Seven Layered Pharmaceutical Composition Comprising the Active Substance Carvedilol

The first layer comprises excipients that cause expansion, the second of carvedilol and controlled release excipients, the third layer of controlled release lag excipients, the fourth layer of carvedilol and controlled release excipients, the fifth layer of controlled release lag excipients, the sixth layer of carvedilol and controlled release excipients and the seventh layer of excipients that cause expansion. See FIG. 7 for an example of the distribution of the different layers.

(IV) Total Ingredients for the Two Tablet Layers Comprising Excipients that Cause Expansion (Layers One and Seven):

| | |
|---|---|
| Microcrystalline cellulose (particle size 20-100 μM) | 28.6 |

### (V) Ingredients for the Controlled Release Tablet Layer Comprising API (Layer Two, Four and Six):

| | |
|---|---|
| Crosslinked hydroxypropylmethylcellulose | 9.7 |
| Methylcellulose | 2.3 |
| Carvedilol | 30 |
| Citric acid | 0.8 |

### (VI) Ingredients for the Controlled Release Lag Tablet Layer Comprising Excipients (Layer Three and Five):

| | |
|---|---|
| Crosslinked hydroxypropylmethylcellulose | 22.6 |
| Methylcellulose | 6 |

Mixtures (IV), (V) and (VI) are blended separately as described in Example 2. Due to the seven different layers, the amount filled in the tablet machine for each layer is accordingly smaller to arrive at a total tablet size that equals the monolayer tablet produced as disclosed in Example 1. Hence the total amount of API in the tablets described in examples 1-3 is the same.

The seven layered tablet is produced by successively compressing each layer soft and short one after the other. When the final layer is filled into the tablet machine the whole tablet is pressed at 2500 psi for 30 seconds on a Carver Press with a conventional circular flat faced tablet punch. The tablet is coated in a conventional coating pan with a 20% w/w solution of polyvinylchloride in tetrahydrofuran (IHF). This may provide a pharmaceutical composition with approximately zero order release of the API.

### Examples 4-7

Because a wide variety of processes may be utilized and products produced according to the invention it is understood that the following experiments are merely examples of the experiments actually conducted and how the present invention may be utilised.

Formulation of tablet cores

| | Amount | Sieve mesh |
|---|---|---|
| Excipient | (weight %) | size (μm) |
| Caffeine granules (BASF AG) | 25 | 700 |
| Tablettose 70 (Molkerei Meggle Wasserburg | 39 | 700 |
| GmbH & Co. KG) | | |
| Titanium Dioxide (H. N. Fussgaard A/S) | 1 | 150 |
| Polyethylen Glycol 20 000 (Merck eurolab) | 33 | 700 |
| Macrogol Stearate | 2 | 700 |

### Manufacturing of Tablet Cores

Macrogol Stearate and 15% of Polyethylen Glycol were melted together in a water bath. This mixture was cooled down by placement in a fridge. The mixture was grated by using a grater and sieved through a sieve with a 700 μm mesh size. Polyethylen Glycol 20 000 was sieved using an oscillating rotor with a mesh size of 630 μm. The remaining excipients were sieved through a mesh with a size as indicated in the table.

All excipients were weighed and blended together by hand in the following order: Titanium Dioxide was mixed together with the same volume of Tablettose 70. More Tablettose 70 was gradually added. Polyethylen Glycol 20 000 was added followed by caffeine granules and then the previously prepared mixture of Polyethylen Glycol and Macrogol Stearate was added. This was then mixed in a food processor (CombiMax 600, Braun) for 1.5 minutes.

Tablets were compressed with a single-punch Diaf Excenter tabletting machine (TM 20, Diaf) by using 5 mm tablet die punches (flat-faced at both ends in order to obtain tablets with sharp edges). The position of the lower punch was set to as low as possible in order to produce circular cylindrical tablet cores with two sharp edges.

Formula of coating fluids, amounts of
excipients are indicated in weight %.

| | Formulation | | | |
|---|---|---|---|---|
| Excipients | I | II | III | IV |
| Ethylcellulose - Ethocel 20 | 1.75 | 1.75 | 1.75 | 1.75 |
| Premium (The Dow Chemical | | | | |
| Group) | | | | |
| Sucrose (Danisco Sugar A/S) | 1.1 | 1.1 | 1.1 | 1.1 |
| Citroflex 4 (Tributyl O- | 0.15 | — | — | 0.15 |
| acetylcitrate, 98%) (Sigma | | | | |
| Aldrich Inc.) | | | | |
| Tartrazin | 0.16 | 0.01 | 0.01 | — |
| Iron oxide - Sicovit ® Brown | — | — | — | 0.045 |
| 70 E-172 (BASF AG) | | | | |
| Ethanol 96% (Kemetyl A/S) | Ad 100 | Ad 100 | Ad 100 | Ad 100 |
| Initial weight of tablet | 50.04 | 50.11 | 50.17 | 50.12 |
| cores (g) | | | | |
| Amount of coating fluid | 83.6 | 87.8 | 95.4 | 129.8 |
| applied (g) | | | | |

### Example 4

### Coating of Tablet Cores

The coating fluid formulation no. I was used in this example. Ethyl cellulose and sucrose were mixed with ethanol. The obtained mixture was stirred with a magnetic stirrer until the coating experiment. To this mixture a colouring agent (Tartrazin) was added to easily identify uncoated areas and further mixing was undertaken. Citroflex-4 (plasticizer) was added about 1 hour before coating. The coating fluid was stirred continuously with a magnetic stirrer throughout the coating process.

The coating fluid was sprayed on the tablet cores (using a fluidized bed coater with top-spray insert) (CombiCoata, Model CC1/lab)(Niro Atomizer) to form a coat around the tablet cores.

The internal diameter of the nozzle was one mm and the position of the nozzle insert was 4 (arbitrary units of the device), the spray pressure was 1.2 bar. The coating fluid was supplied with the help of a pump. The pump was a Watson Marlow (Model 503 S), with 3.2 mm diameter tubing. The average spray rate during the coating was around 6 g/min.

During coating the inlet air temperature was manually adjusted to keep the outlet air temperature at 44-46° C. The airflow rate was adjusted manually during the whole process to keep the tablets in fluidisation, airflow rate at the start of the process was 5 psi and it varied from 5 psi to 6.5 psi.

A drying step was performed 8 min after initiation of the coating process to allow for the following sampling. After sampling, coating was continued until all of the coating fluid was applied to the tablets. The duration of the coating process was 18 minutes and 50 seconds. This includes a drying step during the coating process as well time used for dismantling and mantling of the coater during sampling. The whole surface of the tablet was coated.

Coating was followed by drying. For drying the airflow rate was 6.2 psi and the inlet air temperature was adjusted in order to have an outlet air temperature of around 50° C. In this example, the final drying step was 5 minutes and 10 seconds.

During drying, the sharp tablet edges were removed. This happened due to the attrition forces in the product container, where the tablets were hitting each other and the product container (walls, bottom of the container, nozzle, etc.). After drying, the tablet had a coating, which was thinner or not present in areas over the edges. These two areas each formed a continuous, uncoated (or thinly coated) loop. This allowed subsequent exposure of parts of the tablet to the aqueous phase. The parts of the coating within each loop became separate and fell off from the tablet after contacting with an aqueous phase, thereby establishing an erosion surface at each end of the tablet.

### Example 5

### Coating of Tablet Cores

The coating fluid formulation no. II was used in this example, wherein no plasticizer was added. Ethyl cellulose and sucrose were mixed with ethanol. The obtained mixture was stirred with a magnetic stirrer until the coating experiment. To this mixture a colouring agent (Tartrazin) was added to easily identify uncoated areas and further mixing was undertaken. The coating fluid was stirred continuously with a magnetic stirrer throughout the coating process.

The coating fluid was sprayed on the tablet cores (using a fluidized bed coater with top-spray insert) (CombiCoata, Model CC1/lab)(Niro Atomizer) to form a coat around the tablet cores.

The internal diameter of the nozzle was one mm and the position of the nozzle insert was 4 (arbitrary units of the device), the spray pressure was 1.2 bar. The coating fluid was supplied with the help of a pump. The pump was a Watson Marlow (Model 503 S), with 3.2 mm diameter tubing. The average spray rate during the coating was around 6.3 g/min.

During coating the inlet air temperature was manually adjusted to keep the outlet air temperature at 42-46° C. The airflow rate was adjusted manually during the whole process to keep the tablets in fluidisation; the airflow rate at the start of the process was 5 psi and it varied from 5 psi to 5.75 psi.

The duration of the coating process was 14 minutes. The whole surface of the tablet was coated.

The coating was followed by drying. During drying the airflow rate was kept at 5.5 psi and the inlet air temperature was adjusted in order to have an outlet air temperature of around 51-52° C. Drying was continued for a period of 36 minutes, during this period three samples were removed from the product container. The overall drying time was less than 36 minutes as it includes time used for dismantling and mantling of the coater during the samplings.

During drying, the sharp tablet edges were removed. This happened due to the attrition forces in the product container, where the tablets were hitting each other and product container (walls, bottom of the container, nozzle, etc.). After drying the tablet had a coating, where the coating was thinner or not present in areas over the edges. These two areas each formed a continuous, uncoated (or thinly coated) loop. This allowed subsequent exposure of parts of the tablet to the aqueous phase. The parts of the coating within each loop became separate and fell off from the tablet after contacting with an aqueous phase, thereby establishing an erosion surface at each end of the tablet.

### Example 6

### Coating of Tablet Cores

The coating fluid formulation no. III was used in this example, wherein no plasticizer was added. Ethyl cellulose and sucrose were mixed with ethanol. The obtained mixture was stirred with a magnetic stirrer until the coating experiment. To this mixture a colouring agent (Tartrazin) was added to easily identify uncoated areas and further mixing was undertaken. The coating fluid was stirred with a magnetic stirrer continuously throughout the coating process. The coating fluid was sprayed on the tablet cores (using a fluidized bed coater with top-spray insert) (CombiCoata, Model CC1/lab) (Niro Atomizer) to form a coat around the tablet cores.

The internal diameter of the nozzle was one mm and the position of the nozzle insert was 4 (arbitrary units of the device), the spray pressure was 1.2 bar. The coating fluid was supplied with the help of a pump. The pump was a Watson Marlow (Model 503 S), with 3.2 mm diameter tubing. The average spray rate during the coating was around 5.7 g/min.

During coating the inlet air temperature was manually adjusted to keep the outlet air temperature at 40-45° C. The airflow rate was adjusted manually during the whole process to keep the tablets in fluidisation; the airflow rate at the start of the process was 5 psi and it was varied from 5 psi to 6.0 psi.

In this example, cyclic coating-drying approach was applied, the tablets were coated for 5 minutes and this was continued by a drying step for next 5 minutes. This coating-drying cycle was applied 2 times. The third and final coating period was 6 minutes and 45 seconds and this was followed by a drying period of 11 minutes.

During the drying intervals, the sharp tablet edges were removed stepwise. This happened due to the attrition forces in the product container, where the tablets were hitting each other as well as the product container (walls, bottom of the container, nozzle, etc.). After the final drying the tablet had a coating, where the coating was thinner or not present in areas over the edges. These two areas each formed a continuous, uncoated (or thinly coated) loop. This allowed subsequent exposure of parts of the tablet to the aqueous phase. The parts of the coating within each loop became separate and fell off from the tablet after contacting with an aqueous phase, thereby establishing an erosion surface at each end of the tablet.

### Example 7

### Coating of Tablet Cores

The coating fluid formulation no. IV was used in this example wherein the plasticizer was added 30 minutes before coating and iron oxide was used as a colouring agent. Ethyl cellulose and sucrose were mixed with ethanol. The obtained mixture was stirred with a magnetic stirrer until the coating experiment. To this a colouring agent (Sicovit® Brown 70 E-172; a water insoluble colouring agent was chosen to better visualise the coated areas) was added and further mixed. In this example, Citroflex-4, (plasticizer) was added 30 minutes before coating. The coating fluid was stirred continuously throughout the coating process. The coating fluid was sprayed on the tablets (using a fluidized bed coater with top-spray insert) (CombiCoata, Model CC1/lab) (Niro Atomizer) to form a coat around the tablet cores.

The internal diameter of the nozzle was one mm and position of the nozzle insert was 4 (arbitrary units of the device), the spray pressure was 1.2 bar. The coating fluid was supplied with the help of a pump. The pump was a Watson Marlow (Model 503 S), with 3.2 mm diameter tubing. The average spray rate during the coating was around 6.2 g/min.

During coating the inlet air temperature was manually adjusted to keep the outlet air temperature at 40-48° C. Airflow rate was adjusted manually during the whole process to keep the tablets in fluidisation, the airflow rate at the start of the process was 5 psi and it varied from 5 psi to 5.5 psi.

In this example, a cyclic coating-drying approach was applied. Tablets were coated for 5 minutes and this was continued by a drying step for the next 5 minutes. The coating-drying cycle was applied 4 times. The final drying period was 20 minutes. The airflow rate at this stage was increased to 6.0 psi to apply higher attrition forces.

During the drying intervals, the sharp tablet edges were removed stepwise. This happened due to the attrition forces in the product container, where the tablets were hitting each other as well as the product container (walls, bottom of the container, nozzle, etc.). After the final drying the tablet had a coating, where the coating was thinner or not present in areas over the edges. These two areas each formed a continuous, uncoated loop. This allowed subsequent exposure of parts of the tablet to the aqueous phase. The parts of the coating within the loops became separate and fell off from the tablet after contacting with an aqueous phase, thereby establishing an erosion surface at each end of the tablet.

### Dissolution Experiment

The release of caffeine from the coated, cylindrical tablets (n=3), prepared in Example 7, in 500 ml of purified water (European Pharmacopeia quality) at 37° C. with 20 rpm stirring rate was determined by using the paddle method (European Pharmacopeia). A Diso-6 (Kraemer Elektronik Gmbh) dissolution apparatus was used. 10 ml samples were removed from the dissolution vessels, and not replaced, at the following time points: 10, 30, and 60 minutes. Before the first sampling at 10 minutes the coating of the end parts, within the loops defined by uncoated parts over the edges of the tablets, had became separated and had fallen off from the tablets. After one hour all the tablets were disintegrated and the intact cylinder of insoluble coating was floating at the water-air interface on top of the dissolution liquid.

The absorbance of caffeine in removed samples was measured at 273 nm using an Evolution 300 UV-visible spectrophotometer (Thermo Scientific). Release of caffeine from the tablets was determined to be linear, as shown below.

| | Amount of Caffeine released (mg) | | |
|---|---|---|---|
| Time point | Tablet | Tablet | Tablet |
| (min) | no. 1 | no. 2 | no. 3 |
| 10 | 5.593 | 5.434 | 5.698 |
| 30 | 18.765 | 18.968 | 19.729 |
| 60 | 32.662 | 34.792 | 31.748 |

### Examples 8-10

Examples 8-10 provides examples of multilayer tablets. These are trilayer controlled release tablets coated with a pan coater.

### Example 8

Erosion driven trilayer controlled release tablets were prepared using Carver Auto Series Hydraulic press using 0.25 inch flat face tooling. Table I and II provide the composition of the inner and outer cores of the trilayer tablets. The blend for inner core and outer core was essentially the same except that the inner core contained HPMC and Caffeine in addition to the other ingredients. The total tablet weight was 500 mg. Table I below lists the core formulation.

TABLE I
Inner core

| | Composition | Ex. 8 | Ex. 9 | Ex. 10 |
|---|---|---|---|---|
| Ingredient | mg/g | mg/tab | mg/tab | mg/tab |
| Caffeine | 50.00 | 20.00 | 15.00 | 10.00 |
| HPMC K15M | 150.00 | 60.00 | 45.00 | 30.00 |
| Emcompress ® | 742.50 | 297.00 | 222.75 | 148.50 |
| (Dicalcium phosphate) | | | | |
| Explotab ® (Sodium | 50.00 | 20.00 | 15.00 | 10.00 |
| starch glycolate) | | | | |
| Magnesium Stearate | 7.50 | 3.00 | 2.25 | 1.50 |
| Totals (mg) | 1000.00 | 400.00 | 300.00 | 200.00 |

The outer layer formulation is given in Table II. The two outer cores each weight 50 mg with an inner core weight of 400 mg.

TABLE II
Outer core

| | Composition | Ex. 8 | Ex. 9 | Ex. 10 |
|---|---|---|---|---|
| Ingredient | mg/g | mg/tab | mg/tab | mg/tab |
| Emcompress ® | 942.50 | 94.25 | 188.50 | 282.60 |
| (Dicalcium phosphate) | | | | |
| Explotab ® (Sodium | 50.00 | 5.00 | 10.00 | 15.00 |
| starch glycolate) | | | | |
| Magnesium Stearate | 7.50 | 0.75 | 1.50 | 2.40 |
| Inner core | | 400.00 | 300.00 | 200.00 |
| Totals (mg) | 1000.00 | 500.00 | 500.00 | 500.00 |

Tablets were prepared on a carver auto series hydraulic press at a compression force of 2300 lbs at a dwell time of 5 sec. The resulting tablets were an average length=9.12 mm and average diameter=6.42 mm.

Tablets were made as schematically illustrated in FIG. 8 (not to scale). The inner core (3) comprising the active ingredient is sandwiched between two outer cores (15). In this example the weight of the inner core was 400 mg, while the weight of each of the outer cores was 50 mg, i.e. a total of 100 mg.

Tablets prepared as described above were then coated with Eudragit RS PO at 4%, 3% and 2% coating weight gains, see Table III. The coating solution was prepared by adding Eudragit to acetone while stirring slowly. Once all the Eudragit was dissolved, Acetyl Tributyl citrate was added slowly with stirring. The resulting coating solution was stirred for 10 minutes. The coating parameters are listed below.

a. Pump speed=10-12 rpm, Pan speed=20 rpm
b. Nozzle pressure=17 psi, Inlet airflow=27-32 CFM
c. Inlet air T=34° C., Exhaust Air T=30° C.

TABLE III
Coating wt gain 4%

| Ingredient | mg/g | mg/tab |
|---|---|---|
| Tablets | 959.60 | 500.00 |
| Eudragit RS PO | 39.27 | 20.46 |
| Acetyl Tributyl Citrate (ATBC) | 5.89 | 3.07 |
| Acetone** | 519.31 | 270.59 |
| Totals | 1000.00 | 523.53 |

**Evaporates on processing

When the coated trilayer tablets with 2%, 3% and 4% weight gains were placed in water, the end parts did not separate. Even after 2 hours the coated tablets were found to be almost intact. A possible reason for the above result may be the insufficient amount of the outer cores at the two ends of the tablet.

### Example 9

Trilayer layer tablets with HPMC K15M grade as the inner core were prepared using 0.25 inch flat face tooling. Table I list the ingredients for the inner core and Table II list the outer core ingredients.

Tablets were prepared on carver auto series hydraulic press at a compression force of 1000 lbs at a dwell time of 5 sec. The resulting tablets were an average length of 9.10 mm and average diameter of 6.41 mm.

The tablets were made as outlined for Example 8. In the present example the weight of the inner core was 300 mg, while the weight of each of the outer cores was 100 mg, i.e. a total of 200 mg.

Tablets were then coated with Eudragit at 4% theoretical coating weight gains using the ingredients of Table III.

The coating solution was prepared by slowly adding Eudragit RS PO to acetone while stirring slowly. Once all the Eudragit was dissolved, Acetyl Tributyl Citrate was added slowly with stirring. The resulting coating solution was stirred for 10 minutes. The coating solution was applied using the following parameters:

a. Pump speed=12-15 rpm, Pan speed=15 rpm
b. Nozzle pressure=18 psi, Inlet airflow=31-32 CFM
c. Inlet air T=34-35° C., Exhaust Air T=27.8-28.8° C.

Preliminary testing in water showed that the end parts, i.e. the outer cores, fell off only 50%. The contents of the inner core were found to be almost intact (swollen although) after 12 hours.

### Example 10

Trilayer layer tablets with HPMC K15M grade as the inner core were prepared using 0.25 inch flat face tooling. Table I list the ingredients for the inner core and Table II list the outer core ingredients.

Tablets were prepared on carver auto series hydraulic press at a compression force of 1000 lbs at a dwell time of 5 sec. The resulting tablets were an average length of 8.77 mm and average diameter of 6.43 mm.

The tablets were made as outlined for Example 8. In the present example the weight of the inner core was 200 mg, while the weight of each of the outer cores was 150 mg, i.e. a total of 300 mg.

Tablets were then coated with Eudragit at 4% theoretical coating weight gains using the ingredients of Table III. The coating solution was prepared and applied as described for Example 9.

Preliminary testing in water showed that the end parts, i.e. the outer cores, all fell off within about 1 minute. The contents of the inner core were found to be almost intact (swollen although) after 12 hours.

Examples 8-10 indicates how coated tablets may be made, the tablets being shaped as cylinders and having protective ends which falls off on contact with water.

1. A pharmaceutical composition for controlled delivery of at least one active ingredient into an aqueous phase, said pharmaceutical composition comprising:
I. a tablet obtainable by compression, said tablet comprising said at least one active ingredient and optionally excipients;
II. a coating, applied on said tablet, said coating covering at least partof said tablet to impede the release of said at least one active ingredient from at least part of the surface of said tablet, said coating being applied in a manner allowing the release of said at least one active ingredient from said tablet after contacting said pharmaceutical composition with said aqueous phase, establishing one or more degradation surfaces of said tablet;
wherein the derivative of the area, a, of each degradation surface, with respect to time, t, obeys:
$$\frac{\partial a}{\partial t} \geq 0 ,$$
after each degradation surface has been established, and/or during delivery of said at least one active ingredient.

2. A pharmaceutical composition, for controlled delivery of at least one active ingredient into an aqueous phase, said pharmaceutical composition comprising:
III. a tablet, preferably obtainable by compression, said tablet comprising said at least one active ingredient and optionally excipients;
IV. a coating, applied on said tablet, said coating covering at least part of said tablet to impede the release of said at least one active ingredient from at least part of the surface of said tablet, said coating being applied in a manner allowing the release of said at least one active ingredient from said tablet after contacting said pharmaceutical composition with said aqueous phase, establishing one or more degradation surfaces of said tablet;
wherein the derivative of the area, a, of each degradation surface, with respect to time, t, obeys:
$$\frac{\partial a}{\partial t} \geq 0 ,$$
after each degradation surface has been established, and/or during delivery of said at least one active ingredient;
wherein said tablet has at least one sharp edge and a coating in proximity of said sharp edge, said coating being diminished in thickness or not present over said sharp edge.

3. The pharmaceutical composition according to claim 1 or 2, wherein the second derivative of the diameter, d, of each degradation surface, with respect to time, t, obeys:
$$\frac{{\partial }^{2} d}{\partial {t}^{2}} \leq 0 ,$$
after each degradation surface has been established, and/or during delivery of said at least one active ingredient.

4. The pharmaceutical composition according to claim 1 or 2, wherein said tablet is a multilayer tablet.

5. The pharmaceutical composition according to claim 4, wherein at least one layer, comprising at least part of said at least one active ingredient, is not in direct contact with said aqueous phase immediately after said one or more degradation surfaces have been established.

6. The pharmaceutical composition according to claim 1 or 2, wherein said tablet consist of a number of layers selected among 1, 2, 3, 4, 5, 6, 7, 8, 9, 10, 11, 12, 13, 14, 15, 16, 17, 18, 19, 20.

7. The pharmaceutical composition according to claim 1 or 2, wherein said tablet comprises at least 2 layers.

8. The pharmaceutical composition according to claim 1 or 2, wherein said tablet comprises at least 3 layers, two of the layers being positioned at opposite extremes of said tablet.

9. The pharmaceutical composition according to claim 8, wherein said at least one coating partly covers the tablet such that no other part than the layers positioned at opposite extremes are in direct contact with the aqueous phase upon contacting said composition with an aqueous phase.

10. The pharmaceutical composition according to claim 1 or 2, wherein degradation of said tablet or release of said at least one ingredient occurs at one or two degradation surfaces.

11. The pharmaceutical composition according to claim 1 or 2, wherein the tablet has parallel sides, preferably between two degradation surfaces.

12. The pharmaceutical composition according to claim 1 or 2, wherein the shape of the tablet and/or the composition is substantially cylindrical such as a right circular cylinder, a circular cylinder, an elliptic cylinder or a pseudo elliptical cylinder.

13. The pharmaceutical composition according to claim 12, wherein the shape of the tablet and/or the composition is a hexahedron, such as a parallelepiped, a rhombohedron, a trigonal trapezohedron, a cuboid or a cube.

14. The pharmaceutical composition according to claim 1 or 2, wherein each degradation surface is approximately constant in size.

15. The pharmaceutical composition according to claim 1 or 2, wherein the surface between the tablet and a surrounding aqueous phase, through which said at least one active ingredient is released, is approximately circular, elliptical, or quadrangular.

16. The pharmaceutical composition according to claim 1 or 2, wherein said tablet is obtainable by a process comprising compression, moulding, casting, extrusion, heat melting, melt casting, and/or a thermoplastic process.

17. The pharmaceutical composition according to claim 1 or 2, wherein said tablet is directly obtainable by compression.

18. The pharmaceutical composition according to claim 1 or 2, wherein said coating is applied by spray coating, pan coating, melt coating, fluid bed coating, cyclone coating, film coating, powder coating, or dip coating.

19. The pharmaceutical composition according to claim 1 or 2, wherein at least one surface area of said tablet is not covered by said at least one coating after said pharmaceutical composition has been brought into contact with the aqueous phase, thereby allowing contact between said tablet and the aqueous phase.

20. The pharmaceutical composition according to claim 1 or 2, wherein the pharmaceutical composition is obtained by coating the whole surface of the tablet, followed by removing a part of the surface thereby allowing subsequent exposure of a part of the tablet to the aqueous phase.

21. The pharmaceutical composition according to claim 20, wherein the removal of a part of the surface occurs after contacting said pharmaceutical composition with an aqueous phase.

22. The pharmaceutical composition according to claim 20, wherein the removal of a part of the surface comprises application of mechanical means, such as cutting, planing, grinding, polishing, planishing, graining, or application of a laser; or application of chemical means such as an acid or a base.

23. The pharmaceutical composition according to claim 1 or 2, wherein said tablet has at least one sharp edge.

24. The pharmaceutical composition according to claim 2 or 23, wherein the thickness of said at least one coating is diminished over said at least one sharp edge.

25. The pharmaceutical composition according to claim 1 or 2, wherein the thickness of said at least one coating is diminished in an area.

26. The pharmaceutical composition according to claim 24 or 25, wherein the area wherein the thickness of said at least one coating is diminished defines a substantially continuous loop area, such that the thickness of said coating within and outside of said loop is larger than the thickness of said coating of said loop area.

27. The pharmaceutical composition according to claim 26, wherein the coating within said loop area is approximately convex or concave.

28. The pharmaceutical composition according to claim 27, wherein the coating within said loop area becomes separate from the rest of the pharmaceutical composition upon contact of the pharmaceutical composition with the aqueous phase.

29. The pharmaceutical composition according to claim 28, wherein the coating within said loop area expands or contracts upon contact with the aqueous phase.

30. The pharmaceutical composition according to claim 1 or 2, wherein the tablet comprises a disintegrant in close proximity with the coating, preferably within said loop area of claim 29.

31. The pharmaceutical composition according to claim 1 or 2, wherein the tablet comprises an excipient, which is easily dissolvable in the solvent of said coating, in close proximity with the coating, preferably within said loop area of claim 29.

32. The pharmaceutical composition according to claim 1 or 2, wherein the contact area between said tablet and the aqueous phase is approximately constant during the delivery of said at least one active pharmaceutical.

33. The pharmaceutical composition according to claim 1 or 2, wherein the aqueous phase is Simulated Gastric Fluid or Simulated Intestinal Fluid according to USP 31, at 37° C.

34. The pharmaceutical composition according to claim 1 or 2, wherein the pharmaceutical composition further comprises an enteric coating covering at least part of said tablet.

35. The pharmaceutical composition according to claim 1 or 2, wherein less than 10 weight % of said at least one active ingredient is delivered during testing for 30 minutes in a USP 31 Paddle Apparatus (II) in Simulated Gastric Fluid.

36. The pharmaceutical composition according to claim 1 or 2, wherein the rate of delivery of said at least one active ingredient is approximately zero order upon testing in a USP 31 Paddle Apparatus in Simulated Intestinal Fluid for a period of time selected among 1, 2, 3, 4, 5, 6, 8, 10 and 12 hours.

37. The pharmaceutical composition according to claim 1 or 2, wherein the maximum delivery rate of said at least one active ingredient is later than a time selected among 0.5, 1, 2, 3, 4, 5, 6, 7, 8, 9, 10, 11 and 12 hours upon testing in a USP 31 Paddle Apparatus in Simulated Intestinal Fluid.

38. The pharmaceutical composition according to claim 1 or 2, wherein the maximum delivery rate of said at least one active ingredient is before a time selected among 1, 2, 3, 4, 5, 6, 7, 8, 9, 10, 11, 12 and 18 hours upon testing in a USP 31 Paddle Apparatus in Simulated Intestinal Fluid.

39. The pharmaceutical composition according to claim 1 or 2, providing burst delivery of said at least one active pharmaceutical ingredient.

40. The pharmaceutical composition according to any of the claims 35-39, wherein the testing in Simulated Intestinal Fluid is preceded by placing said pharmaceutical composition in 30 minutes in a USP 31 Paddle Apparatus in Simulated Gastric Fluid.

41. The pharmaceutical composition according to claim 1 or 2, wherein the tablet comprises an erosion rate modifier.

42. The pharmaceutical composition according to claim 1 or 2, wherein the tablet comprises a disintegrant or lubricant.

43. The pharmaceutical composition according to claim 1 or 2, wherein the tablet comprises a number of different active pharmaceutical ingredients selected among 1, 2, 3, 4, 5, 6, 7, 8, 9, and 10.

44. The pharmaceutical composition according to claim 1 or 2, wherein said tablet comprises an excipient selected among starch, clay, cellulose derivatives, gums, and mixtures thereof.

45. The pharmaceutical composition according to claim 1 or 2, wherein said coating comprises a material selected among eudragits, acrylates, cellulose derivatives, ethyl cellulose, polyethylene, polyethylene oxide, polymethane, silicone, rubber, gums, latex, shell lac, glass, ceramics, waxes, carnauba wax, paraffin and mixtures of thereof.

46. The pharmaceutical composition according to claim 1 or 2, for human or animal use.

47. The pharmaceutical composition according to claim 1 or 2, for pain relief, such as chronic pain, treatment of CNS, or related diseases.

48. The pharmaceutical composition according to claim 1 or 2, wherein said at least one ingredient is selected among antibiotics, minerals, or vitamins.

49. The pharmaceutical composition according to claim 1 or 2, for intracavity administration, such as oral, rectal, or vaginal administration.

50. The pharmaceutical composition according to claim 1 or 2, for intradermal, intraperotoneal, or intradermal administration; or administration to the pancreas, the brain; as an implantate; and/or as an injectable tablet.

51. A method for making a pharmaceutical composition for controlled delivery of at least one active ingredient into an aqueous phase, said method comprising:
V. producing a tablet by compression, comprising said at least one active ingredient and optionally excipients;
VI. subsequently providing at least part of said tablet with at least one coating to impede the release of said at least one active ingredient from at least part of the surface of said tablet, said coating being applied in a manner allowing the release of said at least one active ingredient from said tablet after contacting said pharmaceutical composition with said aqueous phase, establishing one or more degradation surfaces of said tablet;
wherein the derivative of the area, a, of each degradation surface, with respect to time, t, obeys:
$$\frac{\partial a}{\partial t} \geq 0 ,$$
after each degradation surface has been established, and/or during delivery 30 of said at least one active ingredient.

52. A method for making a pharmaceutical composition for controlled delivery of at least one active ingredient into an aqueous phase, said method comprising:
VII. Producing a tablet, preferably by compression, said tablet comprising said at least one active ingredient and optionally excipients;
VIII. subsequently coating at least part of said tablet with at least one coating to impede the release of said at least one active ingredient from at least part of the surface of said tablet, said coating being applied in a manner allowing the release of said at least one active ingredient from said tablet after contacting said pharmaceutical composition with said aqueous phase, establishing one or more degradation surfaces of said tablet;
wherein the derivative of the area, a, of each degradation surface, with respect to time, t, obeys:
$$\frac{\partial a}{\partial t} \geq 0 ,$$
after each degradation surface has been established, and/or during delivery of said at least one active ingredient;
and wherein said tablet has at least one sharp edge and a coating in proximity of said sharp edge, said coating being diminished in thickness or not present over said sharp edge.

53. The method according to claim 51 or 52, wherein the second derivative of the diameter, d, of each degradation surface, with respect to time, t, obeys:
$$\frac{{\partial }^{2} d}{\partial {t}^{2}} \leq 0 ,$$
after each degradation surface has been established, and/or during delivery of said at least one active ingredient.

54. The method according to any of claims 51-53, wherein said at least one coating is manufactured as a tube, and said tablet is placed inside said tube, preferably by pushing said tablet into said tube and/or by pulling said tube over said tablet.

55. The method according to claim 54, wherein said tube is shrunk after placing said compressed tablet into said tube, preferably by heat treatment.

56. The method according to claim 51 or 52, wherein said at least one coating is a fluid at the time of application.

57. The method according to tiny of claim 51 or 52, wherein said coating constitutes a tube obtained by casting, preferably by a thermoplastic process, die casting or injection moulding.

58. The method according to claim 51 or 52, wherein a part of the surface of said tablet comprises an excipient, which is not compatible with said coating material, thereby rendering the tablet partly coated upon coating of the tablet.

59. The method according to claim 51 or 52, wherein the pharmaceutical composition is obtained by coating the whole surface of the tablet, followed by removing a part of the surface thereby allowing subsequent exposure of a part of the tablet to the aqueous phase.

60. The pharmaceutical composition obtainable according to claim 51 or 52.

61. The pharmaceutical composition according to or obtainable according to claim 51 or 52, wherein said tablet comprises an active pharmaceutical ingredient selected among morphine, hydrocodone, oxycodone, hydromorphone, and carvedilol, as well as pharmaceutically acceptable salts thereof.
